(19) United States
(12) Patent Application Publication
(10) Pub. No.: US 2026/0207485 A1
(43) Pub. Date: Jul. 23, 2026

(54) CHEMICAL COMPOUND THAT PROMOTES HEALTHY NAIL GROWTH AND CONTRIBUTES TO THE PROMOTION OF THE HAIR CYCLE AND GROWTH AND THE PREVENTION OF HAIR LOSS

(71) Applicant: SYNAPS BIYOTEKNOLOJI ANONIM SIRKETI, Ankara (TR)

(72) Inventors: Nidal TOMAN, Berlin (DE); Canberk YILDIZ, Berlin (DE)

(73) Assignee: SYNAPS BIYOTEKNOLOJI ANONIM SIRKETI, Ankara (TR)

(21) Appl. No.: 19/144,517

(22) PCT Filed: Nov. 30, 2023
(86) PCT No.: PCT/TR2023/051430

§ 371 Date: Jun. 30, 2025

(30) Foreign Application Priority Data
Jan. 19, 2023 (TR) 2023/000675

(51) Int. Cl. A61K 9/00; A61K 31/122; A61K 31/198; A61K 31/4188; A61K 31/4415; A61K 31/455; A61K 31/51; A61K 31/519; A61K 31/525; A61K 31/714; A61K 33/04; A61K 33/30; A61K 33/34; A61K 36/185

(52) U.S. Cl. CPC A61K 9/0053; A61K 9/0029; A61K 31/122; A61K 31/198; A61K 31/4188; A61K 31/4415; A61K 31/455; A61K 31/51; A61K 31/519; A61K 31/525; A61K 31/714; A61K 33/04; A61K 33/30; A61K 33/34; A61K 36/185

(57) ABSTRACT

A chemical compound is provided. The chemical compound provides hair and nail health by providing the biosynthesis of keratin and collagen, which are the building blocks of the hair and nails, and the components necessary for hair and nail health, thanks to the components it includes and the chemical drug substances of these components.

### CROSS REFERENCE TO THE RELATED APPLICATIONS

This application is the national phase entry of International Application No. PCT/TR2023/051430, filed on Nov. 30, 2023, which is based upon and claims priority to Turkish Patent Application No. 2023/000675, filed on Jan. 19, 2023, the entire contents of which are incorporated herein by reference.

### TECHNICAL FIELD

The invention relates to a chemical compound that provides hair and nail health by providing the biosynthesis of keratin and collagen, which are the building blocks of the hair and nails, and other necessary components for hair and nail health, thanks to the components it includes and the chemical drug substances of these components.

### BACKGROUND

As is known in the art, the hair consists of hairs consisting of a type of protein called keratin. The anatomy of the hair includes the hair shaft, the visible part of the hair, and the hair follicle, the structure that surrounds the hair follicle on the skin. The hair shaft consists of dead, keratinized cells packed tightly together. It consists of several layers, including the hair follicle, the papilla, the hair matrix, and the hair bulb. The papilla is a small, nipple-like structure located at the base of the hair follicle that includes the blood vessels that feed the growing hair. The hair matrix is the actively growing part of the hair follicle, where new cells are produced. The hair bulb surrounds the papilla and includes the cells that will turn into the hair shaft. Hair with the aforementioned anatomy has a cycle that constantly enters the growth and loss phases in humans. The hair cycle includes the following phases: the active growth period of the hair known as the anagen phase, the process known as the catagen phase, where the nutrition and growth of the hair stops, the listening phase of the hair known as the telogen phase. Following these, the cycle of the old hair is completed with the “exogenous” phase in which the old hair strands are shed while the formation process of the new hair begins again at the hair follicle in the process defined as “early anagen”. The anagen phase mentioned here lasts for about 2-8 years and ends with the catagen phase, where 2-4 weeks of hair is degraded. The telogen phase starts after said catagen phase and again lasts for 2-4 weeks. Hair loss only occurs with the start of the next anagen phase and the growth of new hair. It is natural for an adult to lose 50-100 hair strands daily as a result of the hair cycle. New hairs are replaced by new ones and the cycle continues in this way.

Different factors such as drug use, radiation, chemotherapy, exposure to chemicals, hormonal and brain factors, thyroid diseases, general or local skin diseases and stress can affect the hair cycle and cause permanent or temporary hair loss.

For a healthy hair structure, natural hair cycle and growth need to be supported. In particular, it is necessary to prevent the loss of amino acids, vitamins, minerals, and protein from stress, malnutrition, or similar conditions that may prevent the natural hair cycle. In the relevant technical field, there are chemical compounds that can be taken into the body with different application types for the elimination of said technical problems.

In the relevant technical field, treatments and chemical compounds generally used to prevent hair loss are used in the functions of eliminating similar deficiencies such as nutrients and/or vitamins and minerals needed by the hair type. While such approaches can help relieve symptoms in the short term, they cannot provide a treatment based on cause-and-effect relationship. Therefore, it is necessary to reveal chemical compounds that can eliminate situations that can prevent hair loss in order to eliminate symptoms and achieve healthy hair for long periods of time in the relevant technical field. For the relevant technical field, studies on the research and development of the most suitable chemical compounds for hair biochemistry should be carried out.

As is known in the art, the nails consist of hardened protein called keratin. Nail anatomy consists of parts including the nail plate, nail matrix, and nail bed. The nail plate is the visible part of the nail located above the nail bed. The nail matrix, also called the matrix unguis or keratogenous membrane, is the active tissue under the nail plate that produces new cells that harden and move outward from the nail root to the nail plate. The nail bed is the skin beneath the nail plate that provides nutrition for the entire nail unit through blood vessels and nerves. The lunula, the visible white crescent-shaped base of the nail, is the visible part of the matrix. Nail growth takes place in the nail matrix. The matrix produces new cells that harden and move outward to form the nail plate. As new cells form, they push the old cells forward, causing the nails to grow. The rate of nail growth can vary depending on factors such as age, sex, and general health, and on average, it grows about 3 mm per month or about 0.1 mm per day in a healthy individual.

However, it is also known that different indications may arise for different doses of the drug substances in the relevant technical field. Acetylsalicylic acid or ASA is one of the best known examples in this regard. The indication for a 100 mg dose of acetylsalicylic acid is quite different from 500 mg. The first dose is used to inhibit platelet aggregation, while the second dose is used for analgesic therapy. Of course, the 500 mg dose inhibits platelet aggregation, but this dose is not suitable for long-term treatment, given the much higher incidence of side effects. It should also be noted that components formed with drug substances that can be effective separately for the same or similar indications can potentially prevent rather than increase the intended reactions. In such combinations, the components can inhibit each other and compete for the same enzymes and coenzymes. Considering all these, the goal of finding an effective treatment is the research and development of chemical compounds that contain the active ingredients in the most appropriate way.

As a result, studies on supporting the natural hair cycle and growth for the relevant technical field, eliminating the technical negativities that cause hair loss, as well as researching and developing chemical compounds containing optimum active ingredients for healthy nail formation and containing these drug substances at optimum dosage values should be carried out.

### SUMMARY

In the relevant technical field, many studies are carried out for the elimination of the negativities caused by technical problems that prevent the natural hair cycle and growth and cause hair loss and for nail health. However, these studies provide short-term solutions for the elimination of said technical problems and symptoms. In order to eliminate said technical problems, it is necessary to reveal chemical compounds that act especially for long periods of time.

The present invention relates to a chemical compound which, thanks to the active ingredients and the chemical compounds including these ingredients, provides a technical effect by acting for a long period of time for the natural hair cycle and growth, and for achieving healthy nails.

Furthermore, the present invention relates to a chemical compound which, thanks to the active ingredients and the chemical compounds including these ingredients, enables the elimination of technical problems that cause hair loss.

The present invention relates to a chemical compound which, thanks to the active ingredients contained therein and the chemical compounds including these ingredients, provides a technical effect of promoting nail health and growth.

### DETAILED DESCRIPTION OF THE EMBODIMENTS

In this detailed description, the subject matter of the invention relates to a chemical compound which, thanks to the components contained therein and the chemical active ingredients thereof, promotes healthy hair cycle and growth, contributes to the prevention of hair loss and at the same time promotes nail health, and is described with examples that will not create any limiting effect for a better understanding of the subject matter.

The chemical compound of the invention includes active ingredients that support the natural hair cycle and growth, as well as eliminating technical problems that cause hair loss and promoting nail health. The active ingredients referred to in the present invention offer the technical solutions and advantages requested from them thanks to the chemical compounds they contain.

The presence of said active ingredients in appropriate dosages in the chemical compound of the invention has also been determined as a result of research and development activities carried out by the present inventors.

The chemical compound mentioned in the present invention may also include some active ingredients whose technical benefits are known in the art. Although the technical benefits of said active ingredients are known individually, studies on what technical negativities and advantages they will provide if these active ingredients are combined have also been determined by the present inventors. It is clear that ensuring that the active ingredients whose technical benefits are known with their separate uses are present in the chemical compound in a stable manner at appropriate dosage values that can provide technical benefits without creating technical negativity will also provide additional technical solutions and advantages for the relevant technical field.

The chemical compound of the invention can be administered orally and/or parenterally. Of course, these applications may have technical solutions and disadvantages against each other. The chemical compound of the present invention can be used as a therapeutic compound in different applications to provide the desired technical solutions and advantages.

The chemical compound includes more than one active ingredients. It is also essential that these active ingredients are included in certain dosage values within the chemical compound.

The chemical compound of the invention includes at least one amino acid. The most important function of these amino acids is to ensure the synthesis or at least support of keratin, which is the basic building block of the hair structure. The chemical compound of the invention preferably includes more than one amino acid component from which technical benefits and solutions are expected.

The chemical compound of the invention includes the L-Arginine amino acid compound. The L-Arginine compound has the closed formula C₆H₁₄N₄O₂, and its CAS number is 74-79-3. The molecular weight is 174.2 g/mol. The function of the L-Arginine compound in the chemical compound is to increase the flow/microcirculation by controlling the “vascular tone” for the hair. Increasing blood flow/microcirculation ensures that the drug substances in the chemical compound of the present invention are delivered to the intended area, especially to the hair follicle. Increasing blood flow/microcirculation also allows the tissue pH value to increase and thus eliminate the formation of acidosis. In addition, better transport of active ingredients to the hair follicle is ensured by encouraging blood circulation.

The chemical compound of the invention includes the L-Lysine amino acid compound. The L-Lysine compound has the closed formula C₆H₁₄N₂O₂, and its CAS number is 70-54-2. The molecular weight is 146.19 g/mol. L-Lysine is included in the chemical compound to activate the enzymes (NO synthase, cystathionine-β-synthase and lyase) necessary to increase hair synthesis. L-Lysine is also involved to support the role of L-Arginine in the bloodstream by slowing the passage of L-Arginine into muscle cells. L-Lysine is included to support the formation of collagen and to naturally block the enzymatic dissolution of collagen and thus prevent the spread of inflammatory, allergic, and carcinomatous processes.

The chemical compound of the invention includes the L-Proline compound. The L-Proline compound has the closed formula C₅H₉NO₂ and its CAS number is 609-36-9. The molecular weight is 115.13 g/mol. L-Proline is included as an important component of collagen protein in the chemical compound. In this way, collagen provides the regeneration of hair and nails as the main component. L-Proline is also included as a precursor component of hydroxyproline. Hydroxyproline is an important component for the structural proteins of collagen and elastin. L-Proline also protects collagen against degradation by blocking collagen-digesting enzymes (collagenases). Since L-Proline should normally be synthesized from L-Glutamic acid and ornithine, its direct administration eliminates the energy-consuming synthesis step.

The chemical compound of the invention includes the L-Cysteine compound. The L-Cysteine compound has the closed formula C₃H₇NO₂S, and its CAS number is 52-90-4. The molecular weight is 121.16 g/mol. L-Cysteine is included in the chemical compound to provide the biosynthesis of keratin.

The chemical compound of the invention includes the L-Cysteine compound, and also includes the L-Methionine compound. The L-Methionine compound has the closed formula CH₅H₁₁NO₂S, and its CAS number is 59-51-8. The molecular weight is 149.21 g/mol. L-Methionine is included in the chemical compound to provide the biosynthesis of keratin with L-Cysteine. Keratin consists of L-cystine, an amino acid containing sulfur obtained by oxidation of two L-Cysteine molecules bound by the disulfide bond. Although the use of L-cystine to promote keratin synthesis is not appropriate because it has no bioavailability, keratin-bound L-cystine is not biochemically appropriate because it consists of L-Cysteine building blocks bound to the polypeptide, not by the combination of free L-cystine.

The chemical compound of the invention includes at least one, several or all of the L-Arginine, L-Lysine, L-Pyrroline, L-Cysteine and/or L-Methionine compounds as amino acid components. In a preferred embodiment, the chemical compound includes L-Arginine, L-Lysine, L-Pyrroline, L-Cysteine and L-Methionine compounds to provide the desired technical solutions and benefits from said components.

The chemical compound of the invention includes at least one polyphenol-providing component. The polyphenol-providing component is preferably an extract of at least one plant. As the polyphenol providing component mentioned herein, at least one of pomegranate, apple, olive, grape, black cumin, red forest fruits, cocoa, soy, radish, zucchini, broccoli, dates, garlic, wheat germ, nettle, ashwagandha root, green tea, turmeric, saffron, ginkgo biloba, red wine or extracts thereof can be used.

The chemical compound of the invention includes pomegranate extract as a polyphenol-providing component. Pomegranate extract acts as an auxiliary radical scavenger in the chemical compound.

The chemical compound of the invention includes at least one mineral as an auxiliary radical scavenger. The chemical compound of the invention includes selenium as a mineral. Selenium also plays a role in the activity of the tyrosinase enzyme, whose technical effects were previously mentioned.

The chemical compound of the invention includes the coenzyme Q10 component as an auxiliary radical scavenger. The values specified for Coenzyme Q10 are critical for the observation of said technical activities, and while these effects are not observed in the lower values, it has been determined that side effects will occur in the upper values.

The chemical compound of the invention includes at least one, several or all of the pomegranate extract, Coenzyme Q10, selenium components as a radical cleaning component. These radical scavengers are essential for the protection of substance donors such as Methionine, as well as their own functions. Methionine, which is otherwise a good radical scavenger, cannot be used in Keratin biosynthesis through L-Cysteine because it will act as a radical scavenger. In addition, the radical scavenger components have an important function in the protection of tyrosinase.

The chemical compound of the invention includes at least one mineral. The chemical compound preferably includes copper and/or zinc as minerals.

The chemical compound of the invention includes copper as a mineral. Copper is included in the chemical compound to perform the function of the tyrosinase enzyme. As is known, the tyrosinase enzyme plays a critical role in the synthesis of melanin in the hair follicle. The decrease in melanin production causes whitening in the hair. Therefore, the chemical compound includes the copper component so that the tyrosinase enzyme can be at the targeted secretion values.

The chemical compound of the invention includes zinc as a mineral. Zinc is included in the chemical compound to ensure that the amino acids in the chemical compound act for a long time. Zinc also plays a role in cell growth and strengthens the immune system by promoting metabolism. Another important function of zinc in the invention is that it modulates the tissue pH value. This is important to treat tissue acidosis in the hair follicle.

The chemical compound of the invention includes at least one vitamin. The chemical compound of the invention includes more than one vitamin. Vitamins act as coenzymes for enzymatic reactions in the chemical compound of the invention. In addition, vitamins are included in the chemical compound as antioxidants. Accordingly, the present invention includes at least one, certain mixtures or all of the vitamins B2, B3, B6, B7, B9 and B12 as vitamins.

As mentioned before, the chemical compound of the invention can be obtained in two forms suitable for intake into the body. These are in the form of liquid for parenteral intake and the other form suitable for oral intake. If they contain drug substances in both forms, the effects specified in this detailed description will be obtained from the chemical compounds.

The chemical compound in the form suitable for oral intake includes the following components;
At least one of the amino acids L-Arginine, L-Lysine, L-Proline, L-Methionine, L-Cysteine,
At least one of the minerals zinc, copper, selenium,
Coenzyme Q component,
polyphenol provider containing at least one of pomegranate, apple, olive, grape, black cumin, red forest fruits, cocoa, soy, radish, zucchini, broccoli, dates, garlic, wheat germ, nettle, ashwagandha root, green tea, turmeric, saffron, ginkgo biloba, red wine or extracts thereof,
vitamin B complex containing vitamins B1, B2, B3, B6, B7, B9 and B12.

The chemical compound in the form suitable for oral intake includes the following components;
L-Arginine in a value between 2000 and 3500 mg,
L-Lysine in a value between 300 and 1000 mg,
L-Proline in a value between 300 and 1000 mg,
L-Methionine in a value between 400 and 1000 mg,
L-Cysteine in a value between 100 and 500 mg,
Zinc in a value between 8 and 12 mg,
Copper in a value between 0.8 and 1.3 mg,
Selenium in a value between 0.02 and 0.08 mg,
Coenzyme Q10 in a value between 100 and 400 mg,
Pomegranate extract in a value between 50 and 200 mg,
Vitamin B1 in a value between 5 and 30 mg,
Vitamin B2 in a value between 50 and 150 mg,
Vitamin B3 in a value between 100 and 300 mg,
Vitamin B6 in a value between 5 and 15 mg,
Vitamin B7 in a value between 2 and 8 mg,
Vitamin B9 in a value between 0.2 and 0.6 mg,
Vitamin B12 in a value between 0.5 and 1.5 mg.

It is critical for the subject matter of the invention that the chemical compound in the form that can be taken orally into the body includes drug compounds in the dosage amounts given above. The values determined here are shared as ranges in order to provide the technical effects given in the detailed description. Above these values, it is possible for the components to show side effects for patients, while below these values, the desired technical effects may not be seen.

The chemical compound in the form suitable for parenteral intake includes the following components;
At least one of the amino acids L-Arginine, L-Lysine, L-Proline, L-Methionine, L-Cysteine,
vitamin B complex containing vitamins B1, B2, B3, B6, B9 and B12.

The chemical compound in the form suitable for parenteral intake includes the following components;
L-Arginine in a value between 1000 and 3000 mg,
L-Lysine in a value between 1000 and 3000 mg,
L-Proline in a value between 500 and 2000 mg,
L-Methionine in a value between 500 and 2000 mg,
L-Cysteine in a value between 200 and 800 mg,
Vitamin B1 in a value between 50 and 200 mg,
Vitamin B2 in a value between 10 and 50 mg,
Vitamin B3 in a value between 100 and 400 mg,
Vitamin B6 in a value between 20 and 100 mg,
Vitamin B9 in a value between 0.2 and 0.8 mg,
Vitamin B12 in a value between 10 and 40 mg.

It is critical for the subject matter of the invention that the chemical compound in the form that can be taken into the body parenterally includes drug compounds in the dosage amounts given above. The values determined here are shared as ranges in order to provide the technical effects given in the detailed description. Above these values, it is possible for the components to show side effects for patients, while below these values, the desired technical effects may not be seen.

The chemical compound suitable for obtaining in the mentioned forms also provides nail health and growth-promoting properties thanks to the drug substances it includes. The factors required for hair and nail health are similar and the chemical compound of the present invention provides said factors for the human body.

The present inventors have presented a chemical compound for preventing the formation of unhealthy nails as well as hair cycle and growth due to not being able to feed more effectively and not taking the drug compounds that need to be taken in their studies for the relevant technical field. Thanks to the drug substances contained in the chemical compound in question and the drug compounds in these drug substances, malnutrition, and the inability to obtain the drug compounds that need to be taken are eliminated for users and it is possible to obtain the necessary nutritional resources for nail and hair health. While the chemical compound of the present invention can be presented in two forms for consumers, the drug substances and their dosage amounts may vary as required by this form. The drug substances and dosage amounts given herein have been obtained as a result of research and development activities for consumers and are a chemical compound with healthy hair cycle-growth and nail health-promoting properties.

The chemical compounds obtained within the scope of this invention enable the biosynthesis of keratin and collagen required for hair and nails.

The chemical compounds obtained within the scope of the invention provide the necessary infrastructure (such as increasing blood circulation, eliminating acidosis, supporting enzymatic reactions) as well as promoting (or supporting and protecting) the biosynthesis of the necessary components in the body in order to ensure hair and nail health. In addition, melanin synthesis is supported by the protection of tyrosinase for the preservation of hair color with the drug substances in the chemical compound. As a result, the chemical compound contributes to the prevention of hair loss and factors such as hair color, thickness, and fullness by supporting the hair cycle and hair growth and contributes to obtaining thick and strong nails by supporting nail growth.

The protection scope of the invention is specified in the appended claims and cannot be strictly limited to those explained in this detailed description for illustrative purposes. It is evident that a person skilled in the art may exhibit similar embodiments in light of the foregoing without departing from the main theme of the invention.

What is claimed is:

1. A chemical compound configured for an oral administration, wherein the chemical compound supports a hair cycle and growth, prevents a hair loss, and promotes a healthy nail growth through active chemical components, and comprises:
L-Arginine, L-Lysine, L-Proline, L-Methionine, and L-Cysteine as amino acids;
at least one selected from the group consisting of zinc, copper, and selenium as a mineral;
a coenzyme Q10;
at least one of pomegranate, apple, olive, grape, black cumin, red forest fruits, cocoa, soy, radish, zucchini, broccoli, dates, garlic, wheat germ, nettle, ashwagandha root, green tea, turmeric, saffron, ginkgo biloba, red wine or extracts of the pomegranate, the apple, the olive, the grape, the black cumin, the red forest fruits, the cocoa, the soy, the radish, the zucchini, the broccoli, the dates, the garlic, the wheat germ, the nettle, the ashwagandha root, the green tea, the turmeric, the saffron, the ginkgo biloba, or the red wine as a polyphenol provider; and
at least one of vitamin B1, vitamin B2, vitamin B3, vitamin B6, vitamin B7, vitamin B9, and vitamin B12.

2. (canceled)

3. (canceled)

4. The chemical compound according to claim 1, wherein the chemical compound comprises the extracts of the pomegranate as the polyphenol provider.

5. The chemical compound according to claim 1, wherein the chemical compound comprises the vitamin B1, the vitamin B2, the vitamin B3, the vitamin B6, the vitamin B7, the vitamin B9, and the vitamin B12.

6. The chemical compound according to claim 1, wherein the chemical compound comprises the active chemical components as follows:
the L-Arginine in a value between 2000 and 3500 mg,
the L-Lysine in a value between 300 and 1000 mg,
the L-Proline in a value between 300 and 1000 mg,
the L-Methionine in a value between 400 and 1000 mg,
the L-Cysteine in a value between 100 and 500 mg,
the zinc in a value between 8 and 12 mg,
the copper in a value between 0.8 and 1.3 mg,
the selenium in a value between 0.02 and 0.08 mg,
the coenzyme Q10 in a value between 100 and 400 mg,
the extracts of the pomegranate in a value between 50 and 200 mg,
the vitamin B1 in a value between 5 and 30 mg,
the vitamin B2 in a value between 50 and 150 mg,
the vitamin B3 in a value between 100 and 300 mg,
the vitamin B6 in a value between 5 and 15 mg,
the vitamin B7 in a value between 2 and 8 mg,
the vitamin B9 in a value between 0.2 and 0.6 mg, and
the vitamin B12 in a value between 0.5 and 1.5 mg.

7. A chemical compound in a liquid form configured for a parenteral administration, wherein the chemical compound supports a hair cycle and growth hair, prevents a hair loss, and promotes a healthy nail growth through active chemical components, and comprises:
L-Arginine, L-Lysine, L-Proline, L-Methionine, and L-Cysteine as amino acids; and
at least one of vitamin B1, vitamin B2, vitamin B3, vitamin B6, vitamin B7, vitamin B9, and vitamin B12.

8. (canceled)

9. The chemical compound according to claim 7, wherein the chemical compound comprises the vitamin B1, the vitamin B2, the vitamin B3, the vitamin B6, the vitamin B9, and the vitamin B12.

10. The chemical compound according to claim 7, wherein the chemical compound comprises the active chemical components as follows:
the L-Arginine in a value between 1000 and 3000 mg,
the L-Lysine in a value between 1000 and 3000 mg,
the L-Proline in a value between 500 and 2000 mg,
the L-Methionine in a value between 500 and 2000 mg,
the L-Cysteine in a value between 200 and 800 mg,
the vitamin B1 in a value between 50 and 200 mg,
the vitamin B2 in a value between 10 and 50 mg,
the vitamin B3 in a value between 100 and 400 mg,
the vitamin B6 in a value between 20 and 100 mg,
the vitamin B9 in a value between 0.2 and 0.8 mg, and
the vitamin B12 in a value between 10 and 40 mg.

11. The chemical compound according to claim 4, wherein the chemical compound comprises the vitamin B1, the vitamin B2, the vitamin B3, the vitamin B6, the vitamin B7, the vitamin B9, and the vitamin B12.

12. The chemical compound according to claim 4, wherein the chemical compound comprises the active chemical components as follows:
the L-Arginine in a value between 2000 and 3500 mg,
the L-Lysine in a value between 300 and 1000 mg,
the L-Proline in a value between 300 and 1000 mg,
the L-Methionine in a value between 400 and 1000 mg,
the L-Cysteine in a value between 100 and 500 mg,
the zinc in a value between 8 and 12 mg,
the copper in a value between 0.8 and 1.3 mg,
the selenium in a value between 0.02 and 0.08 mg,
the coenzyme Q10 in a value between 100 and 400 mg,
the extracts of the pomegranate in a value between 50 and 200 mg,
the vitamin B1 in a value between 5 and 30 mg,
the vitamin B2 in a value between 50 and 150 mg,
the vitamin B3 in a value between 100 and 300 mg,
the vitamin B6 in a value between 5 and 15 mg,
the vitamin B7 in a value between 2 and 8 mg,
the vitamin B9 in a value between 0.2 and 0.6 mg, and
the vitamin B12 in a value between 0.5 and 1.5 mg.

13. The chemical compound according to claim 5, wherein the chemical compound comprises the active chemical components as follows:
the L-Arginine in a value between 2000 and 3500 mg,
the L-Lysine in a value between 300 and 1000 mg,
the L-Proline in a value between 300 and 1000 mg,
the L-Methionine in a value between 400 and 1000 mg,
the L-Cysteine in a value between 100 and 500 mg,
the zinc in a value between 8 and 12 mg,
the copper in a value between 0.8 and 1.3 mg,
the selenium in a value between 0.02 and 0.08 mg,
the coenzyme Q10 in a value between 100 and 400 mg,
the extracts of the pomegranate in a value between 50 and 200 mg,
the vitamin B1 in a value between 5 and 30 mg,
the vitamin B2 in a value between 50 and 150 mg,
the vitamin B3 in a value between 100 and 300 mg,
the vitamin B6 in a value between 5 and 15 mg,
the vitamin B7 in a value between 2 and 8 mg,
the vitamin B9 in a value between 0.2 and 0.6 mg, and
the vitamin B12 in a value between 0.5 and 1.5 mg.

14. The chemical compound according to claim 9, wherein the chemical compound comprises the active chemical components as follows:
the L-Arginine in a value between 1000 and 3000 mg,
the L-Lysine in a value between 1000 and 3000 mg,
the L-Proline in a value between 500 and 2000 mg,
the L-Methionine in a value between 500 and 2000 mg,
the L-Cysteine in a value between 200 and 800 mg,
the vitamin B1 in a value between 50 and 200 mg,
the vitamin B2 in a value between 10 and 50 mg,
the vitamin B3 in a value between 100 and 400 mg,
the vitamin B6 in a value between 20 and 100 mg,
the vitamin B9 in a value between 0.2 and 0.8 mg, and
the vitamin B12 in a value between 10 and 40 mg.

15. The chemical compound according to claim 11, wherein the chemical compound comprises the active chemical components as follows:
the L-Arginine in a value between 2000 and 3500 mg,
the L-Lysine in a value between 300 and 1000 mg,
the L-Proline in a value between 300 and 1000 mg,
the L-Methionine in a value between 400 and 1000 mg,
the L-Cysteine in a value between 100 and 500 mg,
the zinc in a value between 8 and 12 mg,
the copper in a value between 0.8 and 1.3 mg,
the selenium in a value between 0.02 and 0.08 mg,
the coenzyme Q10 in a value between 100 and 400 mg,
the extracts of the pomegranate in a value between 50 and 200 mg,
the vitamin B1 in a value between 5 and 30 mg,
the vitamin B2 in a value between 50 and 150 mg,
the vitamin B3 in a value between 100 and 300 mg,
the vitamin B6 in a value between 5 and 15 mg,
the vitamin B7 in a value between 2 and 8 mg,
the vitamin B9 in a value between 0.2 and 0.6 mg, and
the vitamin B12 in a value between 0.5 and 1.5 mg.
